(19) United States
(12) Patent Application Publication
(10) Pub. No.: US 2021/0030995 A1
(43) Pub. Date: Feb. 4, 2021

(54) Heater For Heating Gas and Method of Manufacturing Same

(71) Applicant: Jae-Sang Park, Seoul (KR)

(72) Inventor: Jae-Sang Park, Seoul (KR)

(21) Appl. No.: 16/964,916

(22) PCT Filed: Mar. 20, 2019
(86) PCT No.: PCT/KR2019/003229

§ 371 Date: Jul. 24, 2020

(30) Foreign Application Priority Data
Jan. 26, 2018 (KR) 10-2018-0009901

(51) Int. Cl. A61M 16/10; H05B 3/46; H05B 3/16; H05B 1/02; B21D 28/26

(52) U.S. Cl. CPC A61M 16/1075; H05B 3/46; H05B 3/16; H05B 1/025; A61M 2202/0241; H05B 2203/02; H05B 2203/017; H05B 2203/022; A61M 2205/3653; B21D 28/26

(57) ABSTRACT

The present invention relates to a heater for heating a gas and a method for manufacturing the same for heating the anesthetic gas or the like by heating the heat transfer area to make it more instantaneous at a short distance. To this end, the present invention is made of an insulator and formed by a slit and a plurality of supports to form a hollow cylindrical or polygonal body, and the slit formed in the body and a portion of the hollow inside the body. It includes a heating wire wound to be exposed to, the slit is formed in a spiral in the body, the support is formed in the middle of the slit, characterized in that the heating wire is wound in the spiral in the body. In addition, the method for manufacturing a heater for heating a gas according to the present invention includes a plate preparation step of preparing a rectangular-shaped plate member made of an insulator, and a slit forming a supports while obliquely forming a plurality of slits through the plate member in the vertical direction. Step and the bending shape forming step of bending the plate member formed with the slit in a cylindrical or polygonal column shape to form a body, and a heating wire spirally winding the heating wire so that a part of the heating wire is exposed inside the body by the slit and the support. Characterized in that it comprises a winding step.

### BACKGROUND OF THE INVENTION

### Field of the Invention

The present invention relates to a heater for heating a gas and a method for manufacturing the same, and more specifically, a heater for heating a gas and a method for heating the gas, which instantaneously heats the temperature of the anesthetic gas and the like to an appropriate temperature by expanding the heat transfer area between the anesthetic gas and the heating cable it is about.

### Background of the Related Art

Generally, gas is compressed and stored in a gas container, and when the stored compressed gas is used, the compressed gas is discharged from the gas container at a low temperature. In addition, since gas does not transfer heat as well as solids and liquids, it is necessary to heat the gas instantaneously to a specified temperature within a short distance, and to control the temperature, indirectly heat it through a medium. It is effective to heat the heating element as a method of directly contacting with the gas instead of the heating method.

Oxygen, which causes the patient to breathe through a suction gas tube connected to the anesthesia machine (Ventilator) used in hospitals, has a low temperature, and anesthetic gases such as ethyl ether and trichlorethylene vaporize the liquid. The result is not only very low temperatures, but also drying.

In addition, when performing major surgery on a patient in a hospital, general anesthesia is performed using an anesthesia machine to paralyze the patient's sensation, lose consciousness, and artificially eliminate pain. Ether, trichlorethylene, etc. are used and the anesthetic gas is inhaled to the patient via the suction gas tube.

At this time, the dry and cold oxygen or anesthetic gas induces hypothermia in the patient and damages the cilia of the respiratory tract. Therefore, the suction gas tube of the anesthesia machine should keep cool and dry anesthetic gas and oxygen in the intensive care unit and operating room at the same temperature as the patient's body temperature to prevent the patient's hypothermia and control the damage to the cilia of the respiratory tract. A heating/humidifying device for heating/humidifying at about the same temperature is connected, and anesthetic gas or the like is heated/humidified by this heating/humidifying device.

However, the conventional heating humidifier is a method of generating water vapor by boiling distilled water and heating the anesthetic gas or oxygen with the heat of the generated water vapor, making it difficult to control the humidity required for the patient, and causing obstacles to the function of the humidifier (Ventilator). In addition, there is a problem that requires a heating device to further heat by cooling again in the process of passing through the long gas suction tube connected to the patient.

Therefore, in order to solve these problems, the suction gas tube connected to the anesthesia machine and the patient should be connected as close as possible to the patient, and oxygen or anesthetic gas should be added momentarily at a short distance. Not only does it require a heater that can heat the gas, but in order to control the temperature of the gas accurately, it is necessary to bring the gas into direct contact with the heating cable and increase the heat transfer area between the gas and the heating cable. It is required to develop a heater for heating a gas that has a structure that allows it. Generally, the gas is compressed and stored in a gas container, and when the stored compressed gas is used, the compressed gas is discharged from the gas container at a low temperature. In addition, since gas does not transfer heat as well as solids and liquids, it is necessary to indirectly heat the airframe through a medium in order to heat the aircraft instantaneously within a short distance at a predetermined temperature and adjust the temperature. It is more efficient to heat the heating element as a method of directly contacting the gas, rather than a heating method.

Oxygen that allows the patient to breathe through an inhalation gas tube connected to a ventilator used in hospitals, etc., has a low temperature, and in particular, an anesthetic gas such as ethyl ether and trichloroethylene is obtained by vaporizing a liquid. Not only is it very low, it is also dry.

In addition, when a patient undergoes major surgery in a hospital, general anesthesia is performed using an anesthesia machine to paralyze the patient's perception, lose consciousness, and artificially eliminate pain. Ether, trichlorethylene, etc. are used and the anesthetic gas is inhaled to the patient via the suction gas tube.

At this time, the dry and cold oxygen or anesthetic gas induces hypothermia in the patient, causing damage to the cilia of the respiratory tract. Therefore, the suction gas tube of the anesthesia machine should be provided with cold, dry anesthetic gas or oxygen in the intensive care unit or operating room to prevent hypothermia of the patient and to prevent damage to the cilia of the respiratory tract. A heating/humidifying device for heating/humidifying at the same temperature as the body temperature is connected, and the anesthetic gas is heated/humidified by the heating/humidifying device.

However, the conventional heating humidifier is a method of generating water vapor by boiling distilled water and heating the anesthetic gas or oxygen with the heat of the generated water vapor, making it difficult to control the humidity required for the patient, and causing malfunction in the function of the humidifier (Ventilator) in addition, there is a problem that requires a heating device to further heat by cooling again in the process of passing through the long gas suction tube connected to the patient.

Therefore, in order to solve such a problem, the suction gas tube connected to the anesthesia machine is connected to the patient as close as possible to the patient, and oxygen or anesthetic gas is instantaneously supplied at a short distance. In addition to requiring a heater that can heat the gas, in order to control the temperature of the gas accurately, the gas should be in direct contact with the heating cable and the heat transfer area between the gas and the heating cable should be increased. It is required to develop a heater for heating a gas with a structure that enables the above.

### SUMMARY OF THE INVENTION

The present invention has been devised to solve the above-mentioned problems, and heat the anesthesia gas and oxygen of an anesthesia device used in the operation of a patient in a hospital to be instantaneously heated in a short-distance suction gas tube. It is an object of the present invention to provide a heater for heating a gas having a structure in which a heat transfer contact area between a gas and a heating cable is extended, and a manufacturing method thereof.

The gas heating heater of the present invention for achieving the above object is formed by a plurality of slits and supports to form a hollow cylindrical or polygonal columnar body, and the slits formed on the body and the supports it includes a heating cable wound to be exposed to the inside of the body, the slit is formed to be inclined in a spiral to the body and the support is formed in the middle of the slit, characterized in that the heating cable is wound in a spiral to the body.

In addition, the supports formed on the body is formed at least two per rotation of the slit and can be formed to be inclined at a constant angle in the longitudinal direction when the body is deployed; and the body is a hollow isosceles triangular pillar it can be formed into a shape.

In addition, when the supports formed on the body is divided into seven by forming the seven supports in the vertical direction by deploying the body, the first slit line and the fourth equidistant line and the seven are divided so that the heating cable is wound at an acute angle it is formed at a position where it intersects the third equidistant line, is formed at a position where it meets the third and sixth equidistant lines at the second slit, and is formed at a position where it meets the second and fifth equidistant lines at the third slit. The supports formed in the second slit and the third slit is characterized in that it is formed repeatedly in the longitudinal direction of the body.

In addition, the body may be formed into a hollow cylindrical pillar shape according to the shape of the flow path through which the gas flows, and when the body is developed and divided into seven equal parts in the longitudinal direction, the first, third, fourth, and six equal parts and the line may be bent to form a hollow parallelogram columnar shape, or bend each of the equal lines to produce a heptagonal columnar shape.

Further, the main body is preferably made of a non-conductor to prevent an electrical short circuit when contacting with a heating cable, and in the case of a conductor such as stainless steel or aluminum material, it is covered with an insulating material, and alternatively can be made of an insulator or a non-conductor by anodizing coating.

The heating cable is characterized by being a heating cable such as nickel, aluminum, and copper, which is a pure metal of a Positive Temperature Coefficient (PTC) heating characteristic that prevents a constant current from flowing over a constant current as the temperature increases.

Further, the heating cable is preferably covered with an insulating material in order to prevent an electrical short circuit that may occur between the body and the heating cable or between the heating cables when wound around the body.

In the present invention, a method of manufacturing a heater for gas heating includes a plate preparation step of preparation a rectangular shape plate member made of a non-conductor;
a slit forming step of forming the supports while obliquely forming a plurality of slits to penetrate the plate member in the vertical direction;
a bending shape forming step of bending or rolling the plate member in which the slit is formed into a cylindrical or polygonal columnar shape to form a body shape;
and characterized in that it comprises a heating cable winding step of winding the heating cable in a spiral shape so that a part of the heating cable is exposed inside the body by the slit and the support.

Further, at least two or more supports formed while forming the slit in the plate member in the slit forming step are formed with respect to the slit, and are formed to be inclined in the longitudinal direction of the plate member.

In addition, in the slit forming step, the supports formed while forming the slit in the plate member is formed to form an acute angle between the supports and the supports when the heating cable is wound along the formed slit, and the plate member is divided into seven equal parts in the vertical direction. When it is done, it is formed in the first slit where it meets the first and fourth equidistant lines and the seventh, and in the second slit it is formed where it meets the third and sixth equidistant lines. It is formed in a position where it meets the line, the supports formed in the first slit, the second slit and the three slit is characterized in that it is formed repeatedly in the longitudinal direction of the plate member.

In addition, the plate member that is bent so that the first slit end and the second slit start step that are bent in the bending shape forming step are aligned, when the plate member is divided into seven equal parts in the vertical direction, the third it is characterized in that it is bent on the basis of the even line, the fourth line, and the sixth line, and is bent in the shape of a parallelogram columnar.

In addition, it characterized in that it further comprises a welding step of welding to the adjacent plate member after the bending step.

### APPLICABILITY TO THE INDUSTRY

According to the heater for heating a gas of the present invention and a method for manufacturing the same, there are the following effects.

First, since the heating cable is spirally wound around the main body and exposed to the inside of the main body by the slit and the supports formed in the main body, the anesthetic gas flowing in the gas tube and the heating cable are connected to the tube by directly contacting and transferring heat in the flow path, heat generation efficiency is improved, and at the same time, the heat transfer area is increased, so that anesthesia gas or the like can be heated instantaneously.

Secondly, the shape of the flow path through which the gas flows through the heating element that has firing characteristics, that is, cannot have a certain shape by itself by making the body in various shapes according to the purpose of heating, that is, in the shape of a hollow cylindrical column or parallelogram column, the contact area between the anesthetic gas and the heating line is increased, and more there is an effect that the anesthetic gas etc. can be efficiently heated instantaneously.

Third, according to the manufacturing method of the present invention, there is an effect that it is possible to simply and easily produce a heater for heating a gas having an increased heat transfer area in direct contact with an anesthetic agent gas and a heating cable.

### BRIEF DESCRIPTION OF THE DRAWINGS

The accompanying drawings, which are included to provide a further understanding of the invention and are incorporated in and configure a part of this application, illustrate embodiment(s) of the invention and together with the description serve to explain the principle of the invention. In the drawings;

FIG. 1 is a perspective view of a heater for heating a gas according to Example 1 of the present invention.

FIG. 2 is a side view of a heater for heating a gas according to Example 1 of the present invention.

FIG. 3 is a perspective view of a body in a heater for heating a gas according to Example 1 of the present invention.

FIG. 4 is an exploded view of the body in the gas heating heater according to Example 1 of the present invention.

FIG. 5 is a perspective view showing a heating cable wound by a heater for heating a gas according to Example 1 of the present invention.

FIG. 6 is a perspective view showing a method of manufacturing a heater for heating a gas according to Example 1 of the present invention.

FIG. 7 is a perspective view of a heater for heating a gas according to Example 2 of the present invention.

FIG. 8 is a side view of a heater for heating a gas according to Example 2 of the present invention.

FIG. 9 is a perspective view of the body in the gas heating heater according to Example 2 of the present invention.

FIG. 10 is an exploded view of the body in the gas heating heater according to Example 2 of the present invention.

FIG. 11 is a perspective view showing a heating cable wound by a heater for heating a gas according to Example 2 of the present invention.

FIG. 12 is a perspective view showing a method of manufacturing a heater for heating a gas according to Example 2 of the present invention.

### DETAILED DESCRIPTION OF THE EMBODIMENTS

Hereinafter, the heater for heating a gas of the present invention and a method for manufacturing the same will be described in detail through Examples 1 and 2 with reference to the accompanying drawings.

### Example 1

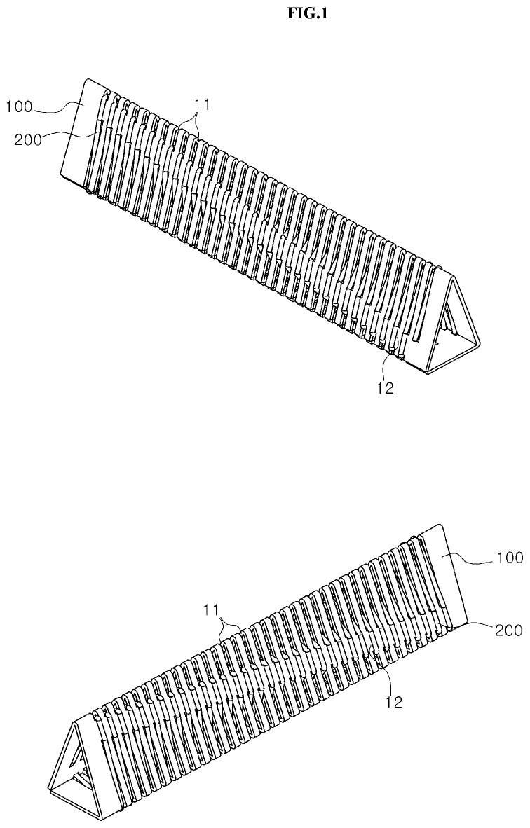

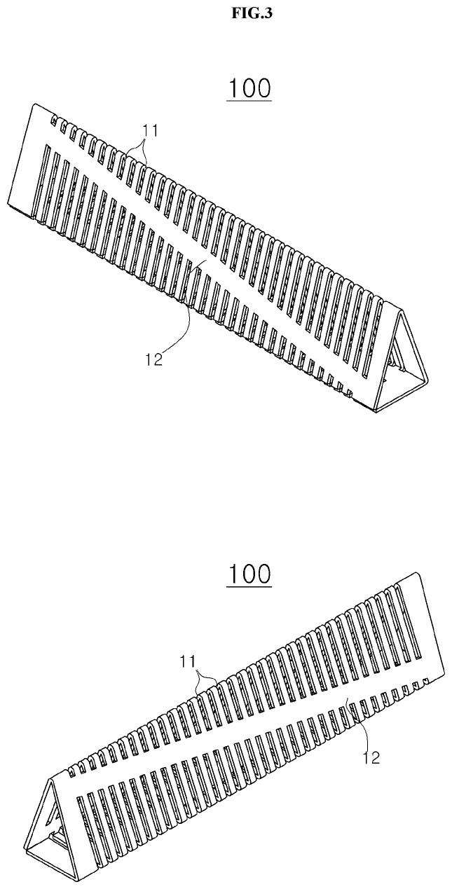

The attached FIGS. 1 to 6 illustrate Example 1 of the present invention, and the heater for heating a gas according to Example 1 of the present invention includes a body 100 and a heating cable 200.

The body 100 is composed of an insulator and is formed in the shape of a hollow isosceles triangular pillar as shown in FIGS. 1 to 3.

In the body 100, a plurality of slits 11 and a plurality of supports 12 are formed.

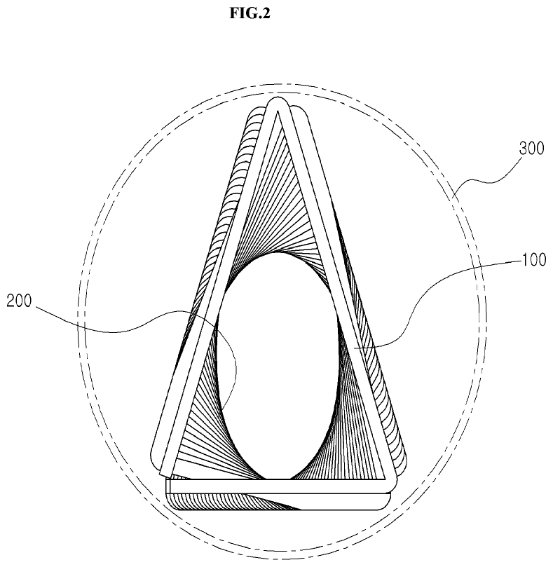

The slits 11, as shown in FIGS. 1 and 2, the heating cable 200 to be described later is formed through the inside of the body 100 in a spiral to be inclined to the body 100, the body 100 to be exposed inside.

And the supports 12 is formed three per rotation of the slits 11, when the heating cable 200 to be described later is wound once, as shown in FIG. 2, the heating cable 200 by the slits 11 is exposed to the inside of the body 100 three times and is supported on the supports 12 to support the heating cable 200 to be described later in a triangular shape.

As shown in FIGS. 1 and 2, the heating cable 200 support by the support 12 is exposed to the inside of the body 100 along the slits 11 of the body 100 and then supported by the supports 12 while being wound, it is spirally wound around the body 100 in a triangular shape.

Since the heating cable 200 is spirally wound on the body 100 by the slits 11 and the supports 12 formed in the body 100 as described above, the gas tube 300 is exposed inside the body 100 and the heat transfer area in which the gas flowing through the heating cable 200 directly contacts is increased, so that the gas and the like can be quickly heated.

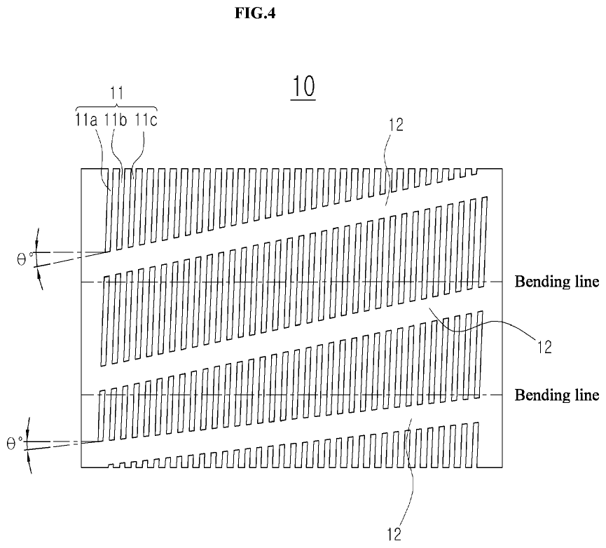

On the other hand, the supports 12 is formed to be inclined at a constant angle (θ) in the longitudinal direction when the body 100 is deployed as shown in FIG. 4.

As described above, since the supports 12 is formed so as to be inclined at a constant angle (θ) in the length direction, the heating cable 200 is continuously wound in a twisted shape as shown in FIG. 1.

In this way, the supports 12 is formed to be inclined at a constant angle (θ), so that the heating cable 200 is wound in a twisted shape, and the gas passing through the gas tube 300 is evenly contacted with the heating cable 200, so that the gas can be heated more quickly.

In addition, the body 100 may be composed of various insulating materials or non-conductors in order to prevent electrical shorts that may occur in contact with the heating cable 200. In the present invention, the body 100 is made of stainless steel or aluminum, and the body 100 is preferably composed of an insulating material by coating an insulating material or by an anodizing coating.

In addition, when the temperature rises due to the characteristic that the temperature rises due to a phase transition when the heating cable 200 reaches a specific temperature region and the electrical resistance increases, the resistance increases and the overcurrent can be prevented. It consists of a Positive Temperature Coefficient (PTC) heating cable 200, such as nickel, aluminum, and copper, which has safer heating characteristics.

The heating cable is made of polyurethane, polyester, or enamel in order to prevent an electrical short that may occur between the body 100 and the heating cable 200 and between the heating cable 200 when wrapped around the body it is preferable to coat with an insulating material such as.

In the first embodiment, three supports 12 are formed per revolution of the slits 11, but more differently, the supports 12 may be formed in the middle part of the slits 11, and the angle formed by the plurality of supports 12 when the body 100 is deployed is a constant angle in the present invention, it is preferable to make the angle formed by the supports 12 to be 9 to 10.

In addition, in the first embodiment, the body 100 is composed of a hollow isosceles triangular pillar, but may alternatively be formed in a hollow cylindrical or polygonal column.

The method for manufacturing the gas heating heater according to Example 1 of the present invention having such a configuration is performed by including a plate preparation step S10, a slit forming step S20, a bending shape forming step S30 and a heating cable winding step S40, after the bending shape forming step S30, a welding step may be further included.

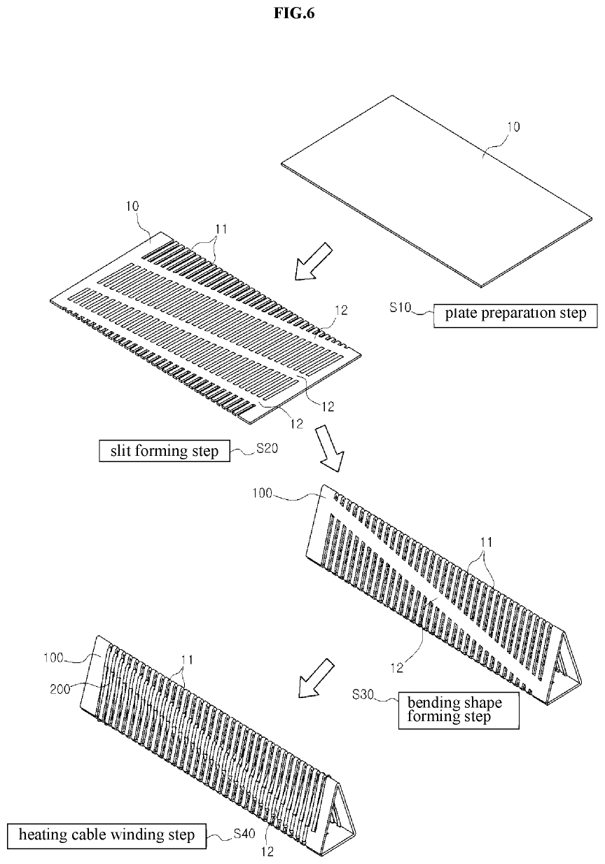

The plate preparation step S10, as shown in FIG. 6, is a step of preparing a rectangular plate member 10 made of an insulating non-conductor, at this time, the plate member 10, it is preferable to prepare an insulating non-conductive material when a conductor such as stainless steel or aluminum is used instead of a nonconductor, additional processing such as plating or anodizing is necessary to make an insulating nonconductor.

The slit forming step S20 is a step of forming the supports 12 while forming a plurality of slits 11 to penetrate the plate member 10 in the vertical direction as shown in FIG. 6.

The plurality of slits 11 formed in the slit forming step S20, so that the upper and lower ends of the adjacent slits 11 in the bending shape forming step S30 to meet each other, that is, described later in the bending shape forming step S30, the slits 11 are formed to be inclined so as to make a spiral shape contact. At this time, the neighboring slits 11 and slits 11 in the plate member 10 are formed at a pitch of one pitch.

In addition, in the slit forming step S20, three supports 12 formed as shown in FIG. 6 are formed with respect to the plurality of slits 11.

After the slits 11 formed in the slit forming step S20 and the heating cable wound in the heating cable winding step S40 described later by the supports 12 are exposed to the inside of the body 100 in a spiral shape forming an acute angle, then supported by the supports 12 and wound in a triangular shape do.

In the step of forming the bending shape, as shown in FIG. 6, the plate member 10 in which the slits 11 are formed is bent into an isosceles triangular pillar shape, and the upper and lower portions of the adjacent slits are in contact with each other, that is, 1 pitch the slits are formed in a spiral shape in the main body 100 so that the heating element can be easily wound in a heating cable winding step S40 described later by manufacturing the slits 11 at intervals.

Meanwhile, the welding step is a step of bending a part or all of the upper and lower ends of the plate member 10 adjacent to each other by bending in the bending shape forming step S30, and the welding step may be omitted if necessary.

The heating cable winding step S40 is a step of spirally winding the heating cable 200 so that a part of the heating cable 200 is exposed to the inside of the body 100 by the slits 11 and the supports 12 as shown in FIG. 6.

At this time, as shown in FIGS. 1 and 2, the heating cable 200 wound in the heating cable winding step S40 is exposed to the inside of the body 100 along the slit of the body 100 and then supported by the supports 12 then, in a triangular shape, the body is spirally wound.

By manufacturing the heater for heating the gas by such a manufacturing method, the heat transfer area in which the gas and the heating cable 200 directly contact in a short gas tube 300 is easily and increased.

On the other hand, the supports 12 formed in the slit forming step S20 is formed to be inclined at a constant angle (θ) in the longitudinal direction when the body 100 is deployed as shown in FIG. 4.

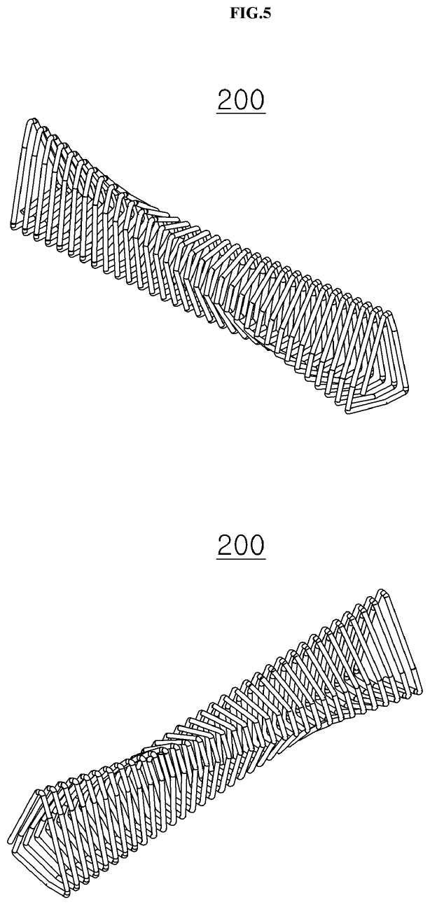

In this way, in the slit forming step S20, as the supports 12 is formed to be inclined at a constant angle (θ) in the longitudinal direction, the heating cable 200 is wound in a triangular shape as shown in FIG. 5.

As described above, the supports 12 is formed to be inclined at a certain angle (θ), so that the heat transfer area between the gas passing through the gas tube 300 and the heating cable 200 is expanded while the heating cable 200 is wound in a spiral shape, so that the anesthetic gas or the like can be instantaneously heated more quickly.

The plate member 10 may be composed of various insulating materials, but in the present invention, it is preferably made of stainless steel or aluminum material, or coated with an insulating material or an anodized coating by an anodizing coating.

When the heating cable 200 wound in the heating cable winding step S40 reaches a specific temperature range and the temperature rises due to a phase transition, the electrical resistance sharply increases it is preferable that the heating cable 200 has a Positive Temperature Coefficient (PTC) characteristic so that the resistance increases together with the increase of the temperature so that a certain amount of current does not flow.

In the Examples 1, three supporting portions 12 formed in the slit forming step S20 are formed per revolution of the slits 11, but alternatively, more parts may be formed in the middle of the slits 11, and when the body is deployed, a plurality of supports portion are formed the constant angle (θ) formed by the addition may be formed at various angles, but is preferably made of approximately 9˜10°.

In addition, in the Example 1, the body 100 formed by being rolled or bent in the bending shape forming step S30 is configured in an isosceles triangular pillar shape, but may alternatively be formed in a hollow cylindrical or polygonal column shape.

### Example 2

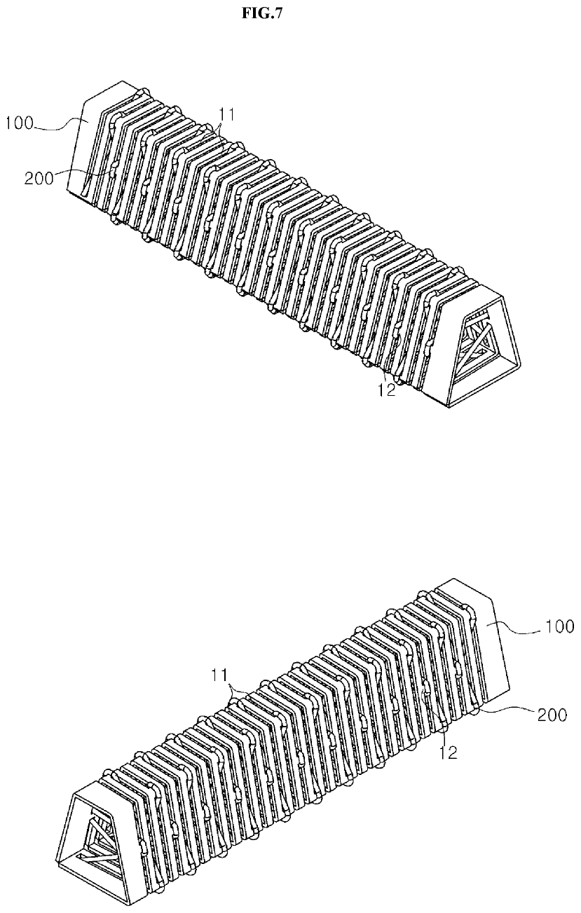

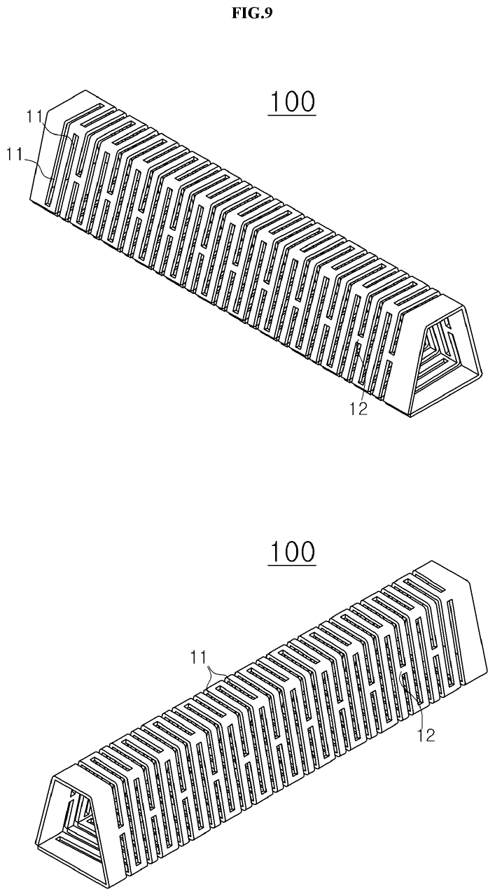

The attached FIGS. 7 to 12 show the second embodiment of the present invention, and the heater for heating the gas according to the second embodiment of the present invention includes the body 100 and the heating wire 200 as in the first embodiment.

The heater for gas heating of the second embodiment is a case in which the heating wire is wound at an acute angle to make the contact area with the gas as wide as possible, and the shape of the body 100 is compared to the heater for gas heating of the first embodiment. It is different, and the support part 12 is formed differently, and accordingly, the heating wire 200 is wound in a different form, which will be described in detail.

The body 100 is made of an insulator and is formed in the shape of a hollow parallelogram column as shown in FIGS. 7 to 9.

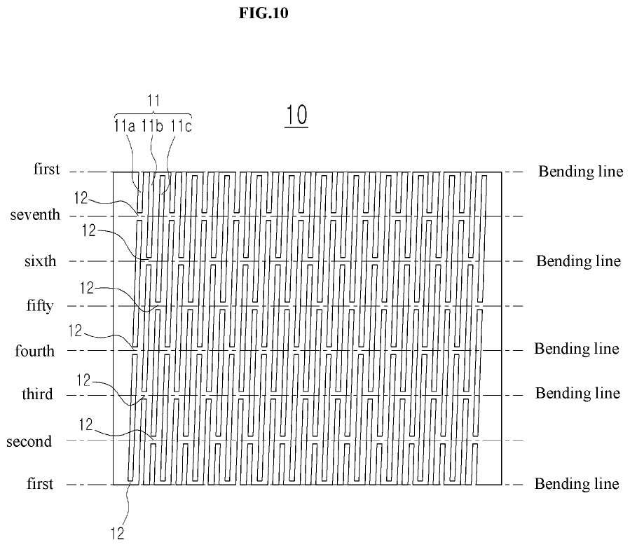

More specifically, when the body 100 is divided into seven equal parts as shown in FIG. 10, the first, third, fourth, and sixth bisecting lines are bent to form a hollow parallelogram columnar shape is formed into.

In addition, as shown in FIG. 10, the supports 12 formed in the body 100 is formed in a position where the first and fourth equidistant lines and the seventh equidistant lines are formed in the first slit 11a, and the third and sixth equidistant lines are formed in the second slit 11b. The third slit 11c is formed by repeatedly repeated in the longitudinal direction of the body 100 at a position where it meets the second and fifth segments.

The heating cable 200 is repeatedly exposed to the inside of the body 100 along the slits 11 of the body 100 as shown in FIGS. 7, 8, and 11 by the support formed repeatedly, and then supported by the support to form a multi-layered star shape the, body is spirally wound.

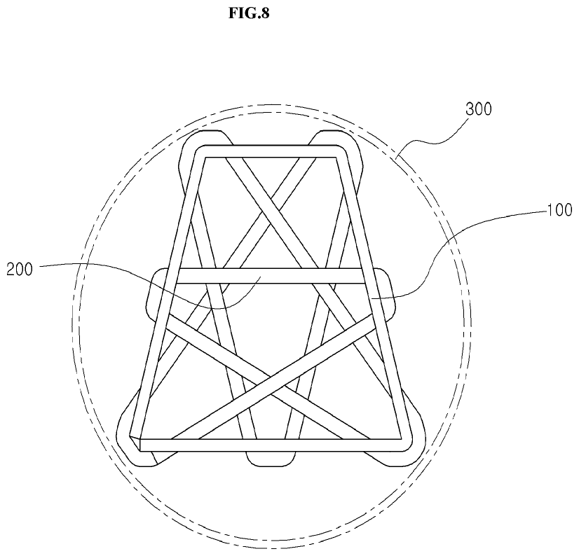

In this way, the body 100 is formed in the shape of a hollow parallelogram and the supports 12 is formed on the slits 11 under a certain arrangement condition, so that the heating cable 200 is wound in a star shape and the gas and heating cable 200 in the gas tube 300 are formed as shown in FIG. 8 by the, increasing the heat transfer area in contact, the gas can be heated more efficiently.

On the other hand, the heating cable 200 is wound along the slits 11, and when the heating cable 200 is wound once, the heating cable 200 is supported on a support formed at a portion where the first and fourth equidistant lines and the seventh equidistant lines are met on the first slit 11a as shown in FIG. 10. When the heating cable 200 is wound two times, it is supported on a support formed at a position where it meets the third and sixth equal lines of the second slit 11b, as shown in FIG. 10, and when the heating cable 200 is wound three times, it is shown in FIG as shown in FIGS. 7, 8 and 11, the third slit 11c is supported by the support formed in the portion of the first slit 11a that meets the first bisecting line, while being supported by the support formed in the second and fifth equidistant lines. It is repeatedly wound in a multi-layered form.

That is, the heating cable 200 is wound around the body 100 three times to form a single star shape at this time, the order in which the heating cable 200 is wound is the first equidistant line, the fourth equidistant line, the seventh equidistant line, the third equidistant line, the sixth equidistant line, the second. It is rolled up repeatedly by making an acute angle in the order of the bisect line, the fifth and the first line.

In this way, the heating cable 200 is wound in a star shape, so that the heating cable 200 directly contacting the gas is evenly distributed inside the gas tube 300, and the heat transfer area is increased, so that the gas can be instantaneously warmed more easily.

In the Example 2, the supports 12 is formed at a uniformly divided position, but may be formed irregularly. In addition, in the Example 2, the body 100 is formed in a hollow parallelogram shape, but in contrast, a hollow shape can also be configured in the form of a cylindrical or polygonal column.

The heater for gas heating of the Example 2, has a different shape of the body 100 and a different supports 12 than the heater for gas heating of the Example 2, and accordingly, the heating cable 200 is wound in a different shape, and the other configuration is the Example 1, since it is the same as the heater for gas, detailed description thereof is omitted.

The method for manufacturing a heater for gas heating according to Embodiment 2 of the present invention includes a plate preparation step S10, a slit forming step S20, a bending shape forming step S30, and a heating wire winding step S40. Here, a welding step of welding an adjacent portion of the body 100 after the bending shape forming step S30 may be further included.

The method for manufacturing the gas heating heater of the Example 2 is different from the method for manufacturing the gas heating heater of the Example 1 in that the supporting portion formed in the slit forming step S20 is different, and the bending the shape of the body 100 that is bent in the shape forming step S30 is different, and the heating cable 200 is wound in another shape in the heating cable winding step S40, which will be described in detail.

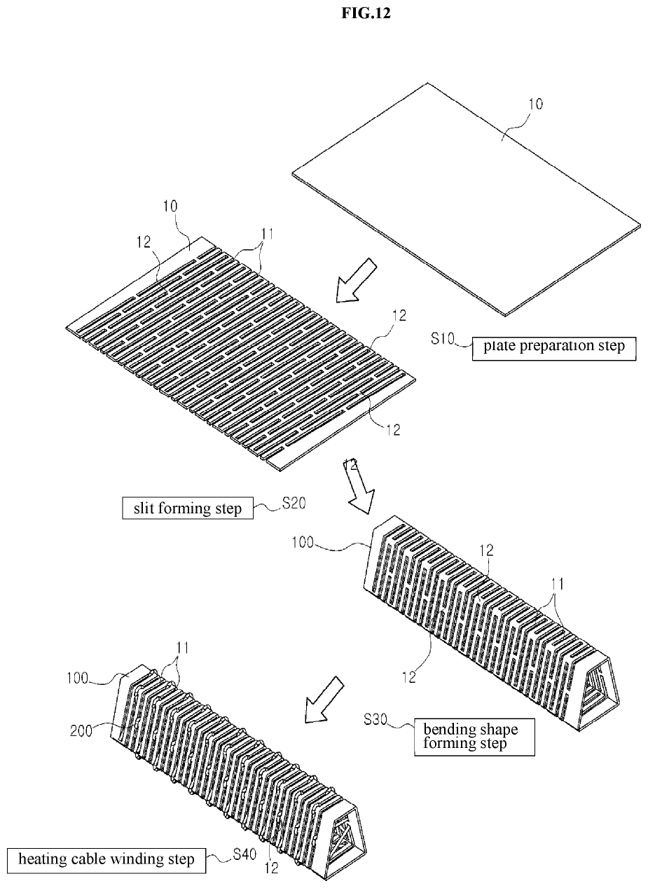

The slit forming step S20, as shown in FIG. 12, the supports 12 formed while forming the slits 11, as shown in FIG. 10, when the plate member 10 is divided into seven equal parts in the length direction, the first slit 11a is formed at the position where it meets the first and the 4th and 7th bisectors, and the second slit 11b is formed at the position where it meets the 3rd and 6th bisectors the third slit 11c are formed at positions where they meet the second bisector and the fifth bisector, and the first slit 11a, the second slit 11b, and the supports 12 formed in the third slit 11c are the plate member 10 it is repeatedly formed in the length direction.

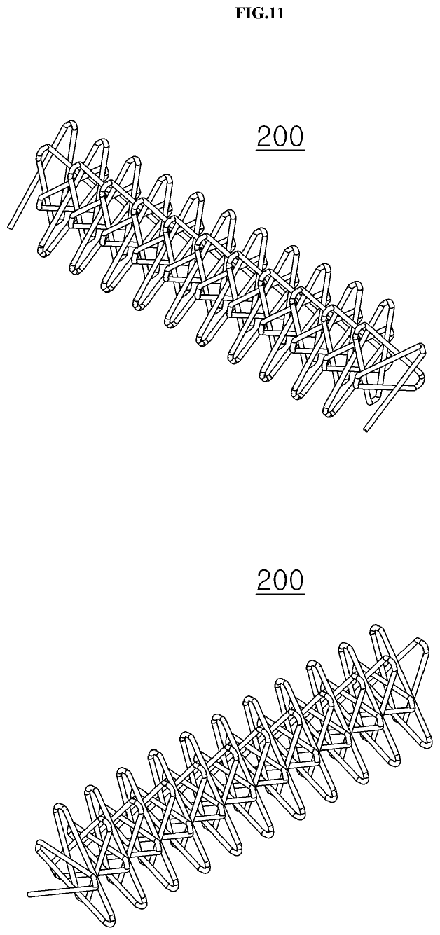

The heating cable by the support formed repeatedly in a predetermined shape on the plate member 10 in the slit forming step S20 is the inside of the body 100 along the slits 11 of the body 100 in the heating cable winding step S40 to be described later as shown in FIGS. 7 and 8 after being exposed, it is supported by the supports 12 and is spirally wound on the body 100 in a star shape as shown in FIG. 11.

In the bending shape forming step S30, as shown in FIG. 12, the plate member 10 having the slits 11 is bent into a parallelogram column to form a body 100 such that the slots are formed spirally while the upper and lower ends of the adjacent slots are in contact with each other it is a step.

More specifically, in the step of forming the bending shape S30, the plate member 10 is divided into 7 equal parts in the length direction as shown in FIG. 10, and bend so that the parallelogram pillar shape main body 100 is formed.

The heating cable winding step S40 is a step of winding the heating cable such that a part of the heating cable 200 is exposed to the inside of the body 100 by the slits 11 and the support as shown in FIG. 12.

At this time, the heating cable 200 wound in the heating cable winding step S40 was exposed to the interior of the body 100 along the slits 11 of the body 100 as shown in FIGS. 7 and 8. While being supported by the supports 12, it is spirally wound on the body 100 in a star shape as shown in FIG. 11.

More specifically, the heating wire wound in the heating cable winding step S40, when wound once, as shown in FIG. 10, the first bisector of the first slit 11a and the fourth bisector and it is supported by a supports 12 formed at the intersection of the seven bisectors. When the heating cable 200 is wound twice, as shown in FIG. 10, it is supported by the support formed at the position where the third and sixth bisectors of the second slit 11b intersect. When the heating cable 200 is wound three times, as shown in FIG. 10, it is supported by the supports 12 formed on the second and fifth bisectors of the third slit 11c. Further, the first slit 11a is supported by the support 12 formed at a portion intersecting with the first bisector and is repeatedly wound in a star shape as shown in FIGS. 7, 8 and 11.

That is, in the heating cable winding step S40, the heating cable 200 is wound around the body 100 three times to form a star shape, wherein the order of the equidistant lines where the heating cables meet is the first equidistant line, the fourth equidistant line, the seventh equidistant line, the third equidistant line, It will be wound repeatedly in the order of 6th, 2nd, 5th, and 1st.

By manufacturing the heated heater according to the manufacturing method of the present invention, it is possible to simply and easily increase the heat transfer area in which the gas and the heating wire 200 directly contact the gas tube 300 at a short distance.

The method for manufacturing a heater for heating a gas in the Example 2 is different from the method for manufacturing a heater for heating a gas in the Example 1 differently, the heating cable 200 is wound in a different form in the heating cable winding step S40, and the other manufacturing steps are the same as in Example 1, so a detailed description thereof will be omitted.

Although the present invention has been described with respect to Examples 1 and 2, the scope of the present invention is not limited to the examples, and various modifications can be made within the scope of the technical idea of the present invention.

What is claimed is:

1. A gas heating heater, comprising;
a body, which is formed in a hollow cylindrical or polygonal columnar shape by forming a plurality of slits and a plurality of supports; and
a heating cable, part of which is wound around the inside of the body to be exposed thereto by the slits formed in the body;
wherein the slits are each formed to be inclined in a spiral shape on the body;
the supports are each formed in the middle part of each slit; and the heating wire is spirally wound to the body.

2. The gas heating heater of claim 1, wherein the plurality of supports formed on the body such that at least two supports are formed at least two per revolution of the slit.

3. The gas heating heater of claim 1, wherein the plurality of support portions formed on the body are formed to incline at a constant angle in the length direction when the body is expanded.

4. The gas heating heater of claim 1, wherein the body is a hollow isosceles triangular pillar.

5. The gas heating heater of claim 1, wherein with respect to the supports formed on the body, when the body is expanded and divided into seven equal parts in a vertical direction, the supports are each formed such that a support is formed in a first slit where it meets with the first, fourth, and seventh equidistant lines; a support is formed in a second slit where it meets with the second and sixth equidistant lines;
and a support is formed in a third slit where it meets with the third and fifth equidistant lines,
wherein the supports formed in the first slit, the second slit, and the third slit are formed repeatedly in a longitudinal direction of the body.

6. The gas heating heater of claim 5, wherein when the body is divided into seven equal parts in a vertical direction by expanding the body, the first, third, fourth, and sixth equidistant bisecting lines are bent be formed in a shape of a hollow parallelogram columnar shape.

7. The heater for heating gas according to claim 1, wherein the body is made of stainless steel or aluminum, and is coated with an insulating material or is made of an insulating non-conductor by anodizing coating.

8. The heating element of claim 1, wherein the heating element is characterized in that the heating cable has a positive temperature coefficient (PTC) characteristic such as nickel, aluminum, and copper, and is coated with an insulating material such as polyurethane, polyester, and enamel.

9. A method for manufacturing a gas heating heater, comprising:
a plate preparation step, in which a rectangular plate member made of an insulator is prepared;
a slit forming step, in which supports are formed on the plate member while obliquely forming a plurality of slits are formed on the plate member to penetrate through the plate member in a vertical direction;
a bending shape forming step, in which the plate member, on which the slits are formed, is bent into a polygonal columnar shape to form the body; and
a heating cable winding step, in which the heating cable is spirally wound such that t the heating cable is exposed to the hollow portion inside the body.

10. The method of claim 9, wherein in the slit forming step, the supports are formed at least two with respect to the slits at least two or more supporting portions of the plate member are formed in the slit in the step of forming the slit.

11. The method of claim 9, wherein the plate member that is bent in the bending shape forming step is bent into a shape of an isosceles triangular column.

12. The method of claim 9, wherein with respect to the supports formed on the plate member in the slit forming step, when the plate member is divided into seven equal parts in a longitudinal direction, the supports are each formed such that a support is formed in a first slit where it meets with the first, fourth, and seventh equidistant lines; a support is formed in a second slit where it meets with the second and sixth equidistant lines; and a support is formed in a third slit where it meets with the second and fifth equidistant lines,
wherein the supports formed in the first slit, the second slit, and the third slit are formed repeatedly in a longitudinal direction of the plate member.

13. The method of claim 9, wherein with respect to the plate member that is bent in the bending shape forming step, when the plate member is divided into seven equal parts in a vertical direction, the plate member is bent into a shape of an isosceles triangular column by bending the bases of the third, fourth, and sixth equidistant lines into a shape of an isosceles triangular column.

14. The method of claim 9, wherein the material of the plate member is made of stainless steel or aluminum, and is coated with an insulating material or is made of an insulating non-conductor by anodizing coating.

15. The method of claim 9, wherein the heating line is a heating cable having a positive temperature coefficient (PTC) characteristic.

16. The method of claim 9, further comprising a welding step in which welding plate member adjacent to each other after the bending shape forming step.

17. The method of claim 9, wherein the neighboring slits formed in the plate member are made at intervals of one pitch.
